(19) United States
(12) Patent Application Publication
(10) Pub. No.: US 2021/0192634 A1
(43) Pub. Date: Jun. 24, 2021

(54) Adjusted Annuity Payments Responsive to Worsening Annuitant Health Conditions

(71) Applicant: Mark S. Lalin, Mountain Lakes, NJ (US)

(72) Inventor: Mark S. Lalin, Mountain Lakes, NJ (US)

(21) Appl. No.: 16/742,190

(22) Filed: Jan. 14, 2020

Related U.S. Application Data
(60) Provisional application No. 62/953,201, filed on Dec. 23, 2019.

(51) Int. Cl. G06Q 40/08; G16H 50/30; G16H 40/67

(52) U.S. Cl. CPC G06Q 40/08; G16H 40/67; G16H 50/30

(57) ABSTRACT

The present invention discloses an investment vehicle in the form of an annuity that increases a payout amount to an investor in failing health. Specifically, the present invention discloses an annuity that increases the annuitant's payout amount either on a dynamic or periodic basis in response to a downward trend in the annuitant's health, so that the annuitant has access to funds that offset rising costs of medical care. The annuitant's health is monitored via dynamically collected parameters such as blood pressure or blood sugar, and/or through periodic medical tests such as blood tests or mammograms.

### BACKGROUND OF THE INVENTION

To begin, our definition of an annuity is a contract between an individual (or entity such as a trust with a named individual annuitant, or corporation holding assets in trust for a specified individual such as a 401k plan) and an insurance company to guarantee a stream of payments to that individual for their life (or in the case of husband and wife or civil union, joint life). These financial vehicles, like other investments, require an initial payment from the beneficiary to the insurance company.

Annuities have existed in various forms throughout history. The Roman Empire had annuity payments for their legionnaires. The Middle Ages had trade guilds in which dues were paid to provide lifetime income for widows and orphans of guild members. Recent history has seen defined benefit pensions from corporations and Social Security payments from modern world governments. These other payment structures can be defined as annuities in their various forms, but this invention relates to the contract between an insurance company and an individual person as previously described and defined. What has not existed and what this invention will seek to solve is for individual annuitant health data to be provided to the insurance company issuing the contract on an ongoing basis to properly price the annuity payments based on the individual's current mortality.

Annuity payments are calculated using actuarial tables of average life expectancies. These statistical samples are taken over a large population of people. Once payments are commenced, the company issuing the contract begins the payments based upon the current prevailing interest rates and the life expectancy of the individual at that specific time, or in the case of a living benefit rider added to the contract, a specified withdrawal percentage. In the latter case, the lifetime withdrawal percentage is based off of a specified baseline stated value.

Recent modern U.S. history (20^{th} century) has seen important developments which have contributed to a rapid increase of the average age of mortality. These factors include, but are not limited to: access to primary medical care for the general population; improved healthcare provided to mothers and babies; availability of immunizations; improvements in motor vehicle safety; clean water supply and waste removal; safer and more nutritious foods; and rapid rate of growth in the general standard of living.

Most recent U.S. history (21^{st} century) has seen and will continue to see more developments which will increase life span, although these are considered to be smaller contributions to longevity. These developments include, but are not limited to: developments and application of new diagnostic, surgical and life sustaining techniques; presence of environmental pollutants; improvements in exercise and nutrition; incidence of violence; isolation and treatment of causes of disease; emergence of new forms of disease; prevalence of smoking and vaping; misuse of drugs and alcohol; changes in the conception of the value of life; and the ability and willingness of our society to pay for the development of new treatments and technologies, and to provide these to the population as a whole.

Most recent history has also seen a dramatic decrease in interest rates worldwide. The combination of low interest rates and increased longevity has led to a decline in the guaranteed lifetime annuity payments from the insurance companies. Longevity has also been accompanied with higher costs of health care maintenance. Whereas a human would have died at 65 in 1980, their working life was for the most part until their death. In 2020, a human has the same working life yet a much longer retirement. Without the corresponding income, and with the increased health care costs associated with longer life spans, these individuals will deplete their savings and be a burden on their families and society. This invention will allow the insurance company to price in the actual mortality of the specific individual, so that they will be guaranteed a higher income in the case of poor health for their life and not be subject to a low interest rate environment and a lengthened overall mortality rate.

For at least these reasons, a need exists for an investment vehicle that increases a payout amount to an investor in failing health. Specifically, a need exists for an annuity that increases the annuitant's payout amount either on a dynamic or period basis in response to a downward trend in the annuitant's health, so that the annuitant has access to funds that offset rising costs of medical care. Such downward trend can be shown through data dynamically collected through a monitor worn by the annuitant, or periodically collected through medical tests conducted at a hospital or a doctor's office.

Existing investment vehicles accommodate neither investors seeking increased income pegged to worsening health, nor investors seeking an annuity with a dynamically-adjusted payout amount. Disclosures of the prior art are instructive in this regard.

For example, U.S. Pat. No. 10,049,407 discloses an insurance policy, as opposed to an annuity, whose premium amount is dynamically recalculated based on certain parameters regarding the insured, including dynamically collected medical information. However, the '407 patent contemplates worsening health data triggering an increase in premium amounts to be paid by the insured, as opposed to additional funds flowing to the insured to assist with medical expenses and/or long-term care.

Similarly, U.S. Pat. No. 8,521,546 discloses a risk assessment model to be used in calculating insurance pricing models. This risk assessment model aggregates dynamically collected medical data associated with the insured, resulting in dynamic risk analysis. Here, too, the invention of the '546 patent does not contemplate annuities, nor does it contemplate increasing income for insured persons in failing health. Rather, the invention of the '546 patent discloses the placement in a higher risk category of an insured person whose dynamically collected data indicates failing health. That person would ostensibly then be charged more for an insurance policy, leaving less money to cover the costs of medical care.

Other investment vehicles are designed to reward insured persons for improving their overall health, as opposed to increasing a payout in response to failing health. For example, U.S. Pat. No. 8,504,461 discloses a financial product where the base rate of return is based on the investor's well-being, with improvements in well-being typically resulting in an increased rate of return. Although the '461 contemplates annuities as compatible with this invention, it does not disclose the dynamic collection of health data via, for example, a health monitor worn by the investor.

Finally, although U.S. Patent Publication No. 2018/0211324 discloses the dynamic adjustment of an annuity based on wellness monitoring of a participant, it does not contemplate increasing the annuity payout amount in response to worsening health. Rather, compliance with the annuitant in a wellness program is correlated to an increased payout amount (and this increase is funded from outside the annuitant's investment). To this end, the need still exists for an annuity capable of dynamically or periodically increasing a payout amount in response to the annuitant's worsening health, in order to better cover the annuitant's increased medical or long-term care expenses.

### SUMMARY OF THE INVENTION

The present invention discloses an investment vehicle in the form of an annuity that increases a payout amount to an investor in failing health. Specifically, the present invention discloses an annuity that increases the annuitant's payout amount either on a dynamic or periodic basis in response to a downward trend in the annuitant's health, so that the annuitant has access to funds that offset rising costs of medical care. The annuitant's health is monitored via dynamically collected parameters such as blood pressure or blood sugar, and/or through periodic medical tests such as blood tests or mammograms.

The invention contemplates an Income Acceleration Factor (IA), that, when triggered, increases the payout amount of the annuity. This Acceleration Factor may be applied to either a single premium immediate annuity, a deferred income annuity, or a fixed annuity, fixed indexed annuity, or variable annuity contract with a guaranteed lifetime income rider attached to the contract. This Acceleration Factor is based upon a particular medical condition that requires additional funds for treatment and/or long-term facility care.

This medical condition is defined as a Total Health Factor (HF_{T}). This (HF_{T}) is “calculated” by integrating the results of standard periodic office medical tests and exams (HF_{PS}) to define and assign an annuitant's baseline health condition (or level) at contract signing. This baseline Health Level is adjusted by periodic office screenings and real-time inputs from medical equipment worn/carried by the annuitant that digitally flows back to an operably-configured server.

The term “real-time inputs” contemplates both current technology and any technology yet to be developed, but intended to continuously monitor specific bodily conditions and digitally transmits collected data back to a central database. Once received by the database, this data is relied upon to continuously or periodically update and adjust the Health Factor, alongside results of the periodic screenings. So, the periodic screening tests are summarized and quantified so as to create a Level of Health. The real-time data is received at the central station and flows into the periodic data permitting an instantaneous, real-time adjustment to the Level of Health.

$$H {F}_{TOTAL} = H {F}_{PERIODIC} + H {F}_{REALTIME}$$

$$H {F}_{PERIODIC} =$$

$${HR}_{REALTIME} = {( MONITORED DATA )}_{THAT FLOWS TO H {F}_{PERIODIC}^{}}^{}$$

The Periodic Screening data sets the Health Factor level at inception and is the data that changes the level over time as additional data are gathered. The Real-Time data can have two functions: 1) to flow back and instantaneously adjust and correct the periodic screening data, and therefore the overall Level of Health; 2) to act independently so as to generate an independent measure of health, especially in critical conditions.

Total Health Factor (HF) is based upon two sets of medical information, i.e. the Health Factor “P.S.” based upon preventative screenings, and the set of medical data gathered from real-time tests, Health Factor “R.T.”.

$$Total Health Factor ( H {F}_{T} )$$

$$( H {F}_{T} ) = {( H {F}_{PS} )}_{Preventative Screenings} + {( H {F}_{RT} )}_{Real \text{-} Time Tests}$$

The HF_{PS} is a compilation of standard, periodic tests, e.g., X-Ray, MRI, Blood Tests, mammograms, colonoscopies, and any new procedure yet to be developed taken in an office setting. These data can be taken on a periodic basis (monthly, quarterly, annually) based upon baseline conditions at sign-up—the “poorer” the individual in terms of his or her health, the more frequent the test update.

HF_{PS}= [symbol] Σ(X-Ray, MRI, BLOOD TESTS, ETC.)

The second element of the HF_{T} is the HF_{RT}, the data generated by the real-time health monitoring systems. These data are gathered in real-time by monitors and equipment worn by the individual and sent back to a system capable of comparing the new data to the past data, to and generating an updated HF_{RT} value instantaneously, or periodically, as desired by the investment vehicle provider.

$$H {F}_{RT} = \sum ( EKG , Blood , other equip . )$$

$$H {F}_{RT} = a level , not a specific value$$

$$H {F}_{TOTAL} = {H {F}_{PS}}_{{Level}_{PS}} + {H {F}_{RT}}_{{Level}_{RT}}$$

These two levels are compared against the individual's baseline data at contract sign-up.

The HF_{T} may be dynamically adjusted based upon the HF_{PS} and HF_{RT}, which in turn are revised based upon Periodic Screening data and Real-Time data received by an operably-configured server. Once the HF_{T} exceeds or falls below a threshold designated by the annuity provider, as appropriate for a given annuitant, the Income Acceleration Factor (IA) is then triggered, increasing payout amounts to the annuitant based upon his or her worsening health. Notably, although the HF_{T} is recalculated in a dynamic fashion, the annuity provider may, but need not, verify whether the IA has been triggered for a given annuitant in a dynamic fashion. That is, the annuity provider may permit the IA to be triggered in “real time,” as the HF_{T} fluctuates along with underlying data. In such a case, the annuitant's payout amount would also be dynamically adjusted. Alternatively, while the HF_{T} may fluctuate dynamically, the annuity provider may permit the IA to be triggered (and the resultant payout amount increased) only on a periodic basis, such as weekly, monthly, quarterly, or annually.

### BRIEF DESCRIPTION OF THE DRAWINGS

FIG. 1 is a diagrammatic view showing the transmission of a plurality of an annuitant's physiological conditions to the central server via a wrist-worn medical monitor. FIG. 1 depicts “real-time” medical readings taken from the annuitant via a monitoring device worn on his wrist, and the wireless transmission of such readings to a centralized server accessible by the investment vehicle provider. This figure is presented as only one permissible embodiment, as the present invention contemplates any wearable monitoring device capable of the continuous collection of biometric readings from the wearer, and the relaying of such readings to a central server.

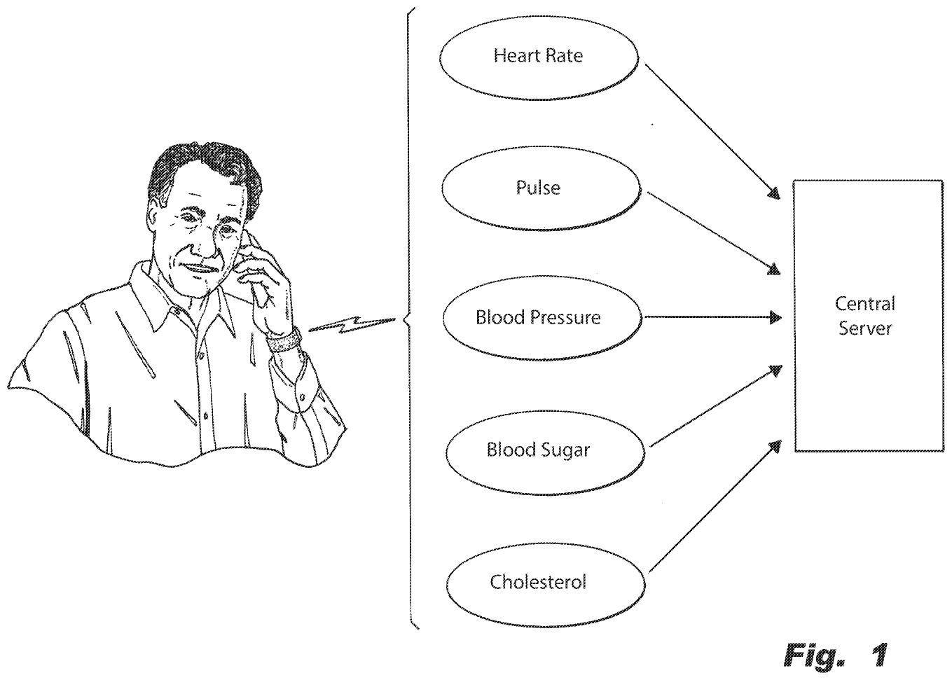

FIG. 2 is a flow chart describing the process as shown in FIG. 1. FIG. 2 depicts a decision tree illustrating one contemplated pathway between the continuous collection of an annuitant's biometric data via a wearable monitoring device and the increase in annuity payout amount to an annuitant corresponding to the latter's worsening health. Per the disclosure of FIG. 2, the present invention contemplates both periodic and dynamic recalculations of this payout amount.

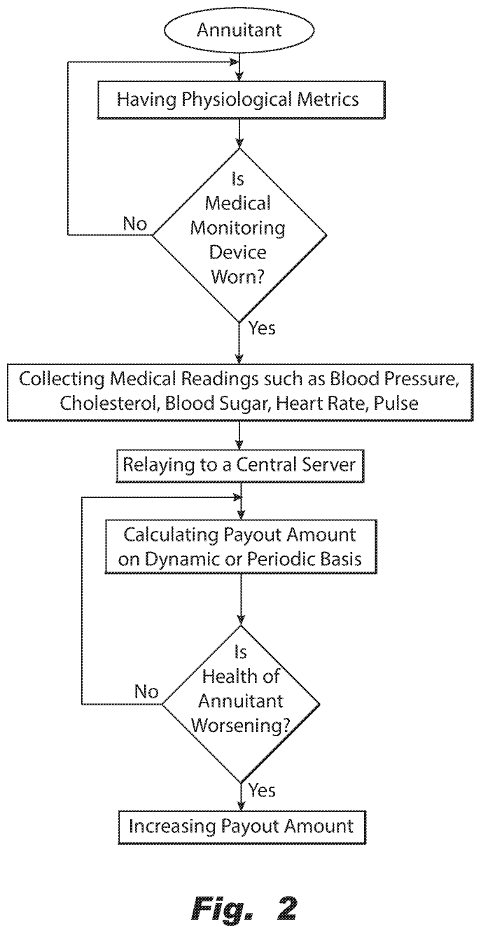

FIG. 3 is a diagrammatic view showing the transmission of a plurality of an annuitant's physiological tests to the central server via a medical scanning apparatus. FIG. 3 depicts a range of periodic medical readings taken from the annuitant via various medical tests, and the transmission of such readings to a centralized server accessible by the investment vehicle provider. This figure presents only a subset of permissible embodiments, as the present invention contemplates all medical tests either in existence or yet to be developed whose results may be interpreted to reflect the overall health condition of the annuitant. Moreover, the present invention contemplates all modes of transmission from a medical laboratory, a doctor's office, or a hospital where such medical tests may be conducted to a central server accessible by the investment vehicle provider.

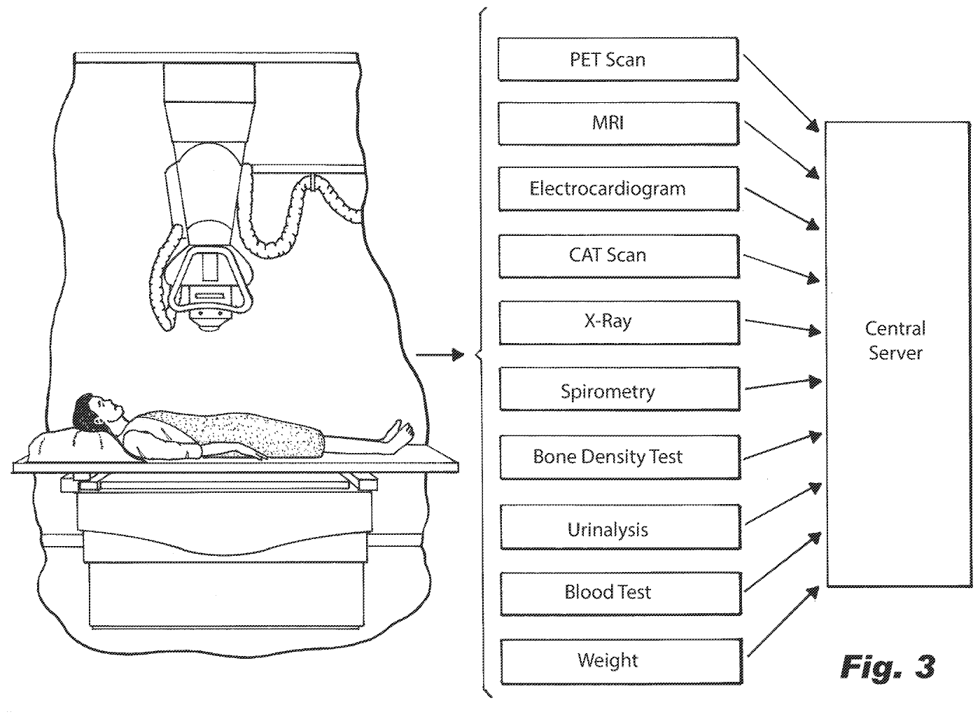

FIG. 4 is a flow chart describing the process as shown in FIG. 3. FIG. 4 depicts a decision tree illustrating one contemplated pathway between the collection of an annuitant's biometric data via periodic medical tests and the increase in annuity payout amount to an annuitant corresponding to the latter's worsening health.

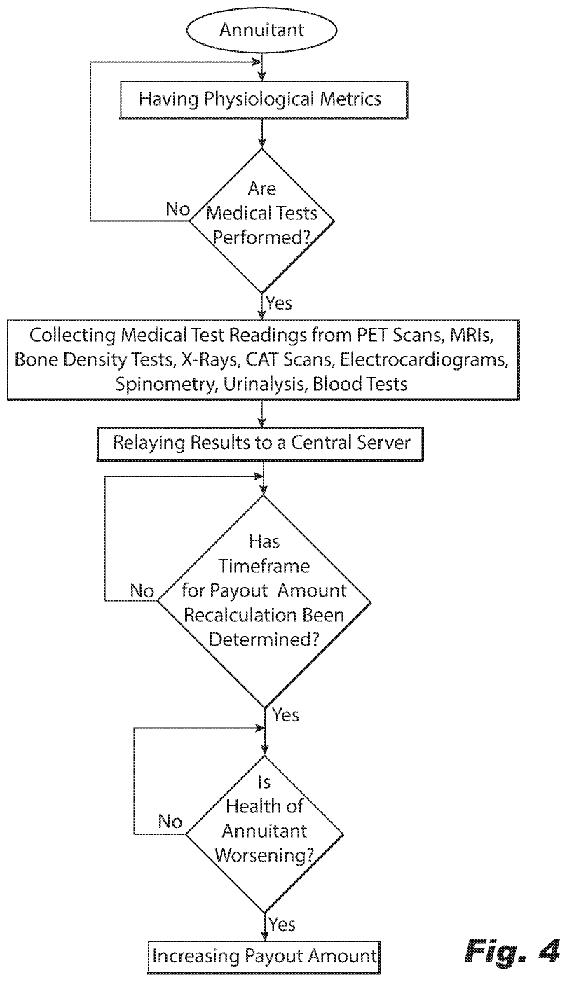

### DESCRIPTION OF EMBODIMENT

In one possible embodiment, annuitant signs his contract at t=0. A physical establishes that screenings and monitors when quantified place him/her in Level 2 of the HF because of high cholesterol and PSA readings. Presuming a six-month update, annuitant's data improves and he moves down the scale to Level 1, and remains constant until year 4 at which time his heart monitor (real-time) indicates irregularities, and his PSA, in combination with the results of an X-Ray or an MRI indicate probable prostate cancer. Numerical values for these issues evidence degradation in the annuitant's health and move him up to Level 2. Medication helps the heart (which is monitored in real time) but the periodically-monitored PSA continues to increase and an MRI indicates probable cancer requiring surgery. This moves the HF up to Level 4 which is the projected threshold for Income Acceleration (IA). Surgery in year 5 shows that the prostate cancer has metastasized and the prognosis is grave. Numerical values for these conditions degrade the HF through the threshold to Level 5 which initiates Income Acceleration to provide additional funds for non-covered medical costs and long-time care facilities.

What is claimed is:

1) A system for dynamically calculating annuity payout amounts comprising:
a. A medical measurement device carried or worn by the annuitant, wherein such medical measurement device continuously collects medical readings associated with one or more of the annuitant's physiological metrics and relays these metrics to a central server system operably connected to the medical measurement device via a communication pathway;
b. Wherein the server system is configured to dynamically recalculate the annuitant's payout amount;
c. Wherein the server's calculation results in the annuitant's payout amount increasing as one or more physiological metrics are interpreted by the server as reflecting the annuitant's worsening health.
d.

[symbol]

2) The system of claim 1 further comprising:
a. The server being operably configured to receive results of medical tests performed on an annuitant;
b. Wherein the server system is configured to dynamically recalculate the annuitant's payout amount in light of the results of the medical tests, and the continuously collected medical readings;
c. Wherein the server's calculation results in the annuitant's payout amount increasing as one or more physiological metrics, and/or the results of one or more medical tests are interpreted by the server as reflecting the annuitant's worsening health.

3) The system of claim 1 wherein the continuously collected medical readings are selected from the group comprising blood pressure and/or pulse, cholesterol, heart rate, and blood sugar levels.

4) The system of claim 2 wherein the medical tests are selected from the group comprising PET scans, MRIs, electrocardiograms, CAT scans, X-rays, weight, lung capacity, bone density tests, urinalysis, and blood tests.

5) A system for periodically calculating annuity payout amounts comprising:
a. A medical measurement device carried or worn by the annuitant, wherein such medical measurement device continuously collects medical readings associated with one or more of the annuitant's physiological metrics and relays these metrics to a central server system operably connected to the medical measurement device via a communication pathway;
b. Wherein the server system is configured to periodically recalculate the annuitant's payout amount;
c. Wherein the server's calculation results in the annuitant's payout amount increasing as one or more physiological metrics are interpreted by the server as reflecting the annuitant's worsening health.
d.

[symbol]

6) The system of claim 5 further comprising:
a. The server being operably configured to receive results of medical tests performed on an annuitant;
b. Wherein the server system is configured to periodically recalculate the annuitant's payout amount in light of the results of the medical tests, and the continuously collected medical readings;
c. Wherein the server's calculation results in the annuitant's payout amount increasing as one or more physiological metric, and/or the results of one or more medical tests are interpreted by the server as reflecting the annuitant's worsening health.

7) The system of claim 5 wherein the continuously collected medical readings are selected from the group comprising blood pressure and/or pulse, cholesterol, heart rate, and blood sugar levels.

8) The system of claim 6 wherein the medical tests are selected from the group comprising PET scans, MRIs, electrocardiograms, CAT scans, X-rays, weight, lung capacity, bone density tests, urinalysis, and blood tests.

9) The system of claim 5 wherein the annuitant's payout amount is recalculated weekly.

10) The system of claim 5 wherein the annuitant's payout amount is recalculated monthly.

11) The system of claim 5 wherein the annuitant's payout amount is recalculated quarterly.

12) The system of claim 5 wherein the annuitant's payout amount is recalculated biannually.

13) The system of claim 5 wherein the annuitant's payout amount is recalculated yearly.

14) The system of claim 6 wherein the annuitant's payout amount is recalculated weekly.

15) The system of claim 6 wherein the annuitant's payout amount is recalculated monthly.

16) The system of claim 6 wherein the annuitant's payout amount is recalculated quarterly.

17)

18) The system of claim 6 wherein the annuitant's payout amount is recalculated biannually.

19) The system of claim 6 wherein the annuitant's payout amount is recalculated yearly.
